(19) United States
(12) Patent Application Publication
(10) Pub. No.: US 2026/0102117 A1
(43) Pub. Date: Apr. 16, 2026

(54) ALCOHOL TEST MANAGEMENT SYSTEM

(71) Applicant: FITZ INC., Zhubei City (TW)

(72) Inventor: WEN-WEI LIN, Zhubei City (TW)

(21) Appl. No.: 19/229,116

(22) Filed: Jun. 5, 2025

(30) Foreign Application Priority Data
Jul. 2, 2024 (TW) 113207103

(51) Int. Cl. A61B 5/00; A61B 5/0205; A61B 5/021; A61B 5/08; A61B 5/097; A61B 5/1171; G06V 10/74; G06V 40/16

(52) U.S. Cl. CPC A61B 5/4845; A61B 5/0205; A61B 5/1176; G06V 10/74; G06V 40/168; G06V 40/172; A61B 5/0077; A61B 5/021; A61B 5/082; A61B 5/097

(57) ABSTRACT

An alcohol test management system includes an alcohol test device, a communication device and an information management device. The alcohol test device respectively generates alcohol test data after completing each alcohol test cycle. The communication device is electrically connected to alcohol test device, to control the alcohol test device in starting each alcohol test cycle, and to receive alcohol test data generated during each alcohol test cycle from alcohol test device. The information management device is electrically connected to communication device, to record each alcohol test data received from the communication device associated with identity data, and to send an activation certificate to the communication device. The communication device operates in the single-user mode upon identifying that the activation certificate corresponds to the single-user mode, and the communication device operates in the multi-user mode upon identifying that the activation certificate corresponds to the multi-user mode.

### CROSS REFERENCE TO RELATED APPLICATIONS

This application claims priority of Application No. 113207103 filed in Taiwan on Jul. 2, 2024 under 35 U.S.C. § 119, the entire contents of both of which are hereby incorporated by reference.

### BACKGROUND OF THE INVENTION

### 1. Field of the Invention

The present invention is related to an alcohol test management system, particularly to an alcohol test management system including an alcohol test device, a communication device, and an information management device.

### 2. Description of the Related Art

Although there are alcohol testers that can be paired with mobile devices, they generally conduct alcohol tests independently, and sync the measurement data to the mobile device after obtaining the alcohol test results. However, this approach cannot confirm the subject of the alcohol test and is prone to cases of false identification or proxy execution of the alcohol test.

Additionally, most alcohol testers do not have single-user mode and multi-user mode. In other words, if a user wants to make the alcohol tester personal, the user needs to purchase a single-user alcohol tester; otherwise, they need to purchase a multi-user model. Once purchased, the user cannot convert a personal alcohol tester to multi-user use, and vice versa. For the manufacturer, this requires the design of two types of alcohol tester hardware, increasing production costs and leading to double the inventory management and sales costs.

### SUMMARY OF THE INVENTION

Upon consideration thereof, the present embodiment proposes an alcohol test management system, including an alcohol test device, a communication device, and an information management device. The alcohol test device generates alcohol test data upon completing each alcohol test cycle. The communication device is electrically connected to the alcohol test device to control the alcohol test device in starting each alcohol test cycle and to receive the alcohol test data generated by each alcohol test cycle, associating each alcohol test data with an identity data. The communication device is configured in a single-user mode or a multi-user mode. In the single-user mode, the identity data associated with each alcohol test data received is bound to the communication device. In the multi-user mode, the identity data associated with each alcohol test data received is received by the alcohol test device or the communication device corresponding to the alcohol test cycle. The information management device is electrically connected to the communication device to record each alcohol test data received from the communication device associated with the corresponding identity data and to send an activation certificate to the communication device. The communication device operates in the single-user mode in response to identifying that the activation certificate corresponds to the single-user mode and operates in the multi-user mode in response to identifying that the activation certificate corresponds to the multi-user mode.

According to the alcohol test management system of the embodiments of the present invention, the operation mode of the communication device can be determined by the activation certificate sent from the information management device, whether it is single-user mode or multi-user mode. Users can change the operation mode of the alcohol test device to single-user or multi-user mode after purchasing the alcohol test device. Therefore, the alcohol test device does not need to distinguish between single-user and multi-user situations with different hardware, which is beneficial for product production, manufacturing, and sales. Additionally, since the alcohol test cycle is initiated by the communication device, the subject's identity can be verified through the communication device during the alcohol test process, providing anti-fraud effects.

### BRIEF DESCRIPTION OF THE DRAWINGS

FIG. 1 is a schematic diagram of the structure of the alcohol test management system according to one embodiment of the present invention.

FIG. 2 is a schematic diagram of the hardware structure of the communication device according to one embodiment of the present invention.

FIG. 3 is a schematic diagram of the software structure of the information management device according to one embodiment of the present invention.

FIG. 4 is a schematic diagram of the hardware structure of the alcohol test device according to one embodiment of the present invention.

FIG. 5 is a schematic diagram of the alcohol test operation according to one embodiment of the present invention.

FIG. 6 is a schematic diagram of the alcohol test operation according to another embodiment of the present invention.

FIG. 7 is a schematic diagram of the cycle of capturing images during the alcohol test operation according to one embodiment of the present invention.

FIG. 8 is a perspective view (one) of the alcohol test device according to one embodiment of the present invention.

FIG. 9 is a perspective view (two) of the alcohol test device according to one embodiment of the present invention.

FIG. 10 is a schematic diagram of the organizational management according to one embodiment of the present invention.

FIG. 11 is a schematic diagram of the structure of the alcohol test management system according to another embodiment of the present invention.

### DETAILED DESCRIPTION OF THE PREFERRED EMBODIMENT

Regarding the term of “coupling” in this specification, it refers to two or more elements making “direct” physical or electrical contact with each other, or making “indirect” physical or electrical contact with each other, thereby allowing interaction between the coupled elements, such as communication and control.

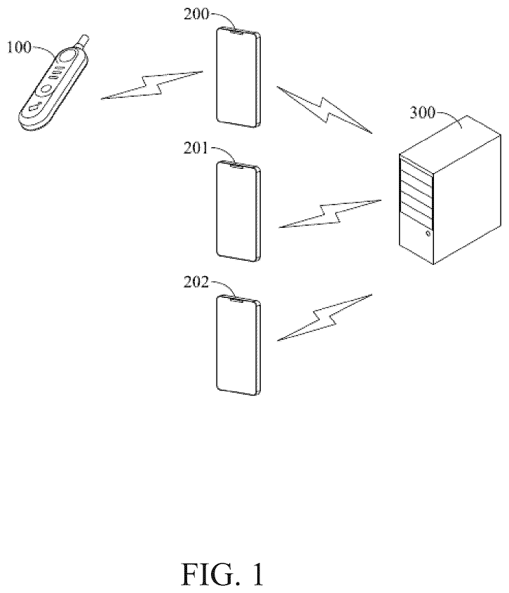

Refer to FIG. 1, which is a schematic diagram of the structure of an embodiment of the alcohol test management system of the present invention. The alcohol test management system includes an alcohol test device 100, a communication device 200, and an information management device 300.

The alcohol test device 100 for performing alcohol tests and generating alcohol test data after each alcohol test cycle. The communication device 200 electrically connected to alcohol test device 100 to control the start of each alcohol test cycle. The communication device 200 receives the alcohol test data generated by each alcohol test cycle from alcohol test device 100 and associates each alcohol test data with an identity data. The information management device 300 electrically connected to communication device 200 to record the alcohol test data received from communication device 200 and corresponding to each identity data. In this way, based on the records, the historical alcohol test data of each identity data can be determined. The communication device 200 is configured in single-user mode or multi-user mode.

In single-user mode, the identity data associated with each alcohol test data received by the communication device 200 is bound to communication device 200. In other words, in single-user mode, the communication device 200 is bound to identity data corresponded to a specific subject, and associates each alcohol test data received with this already bound identity data.

In multi-user mode, the identity data associated with each alcohol test data received by communication device 200 is bound to the identity data corresponded to the alcohol test cycle received by alcohol test device 100 or communication device 200. Specifically, to associate each alcohol test data with the identity of the subject, each alcohol test cycle conducted by a subject is received by alcohol test device 100 or communication device 200, with the identity data corresponded to this subject being received by either alcohol test device 100 or communication device 200. If alcohol test device 100 receives this identity data, it sends the identity data to communication device 200. The communication device 200 associates the identity data it receives with the alcohol test data.

In some embodiments, to facilitate multiple subjects conducting alcohol tests, the alcohol test device 100 does not turn off immediately after completing each alcohol test cycle. Instead, it remains powered on for a period of inactivity (such as 10 minutes) before turning off. This allows the next subject to quickly start the next alcohol test cycle without having to wait for the alcohol test device 100 to power on, connect, and go through other preparation procedures.

The information management device 300 is used to manage the permissions of the communication device 200, which operates in single-user mode or multi-user mode. After undergoing permission authentication (e.g., confirming that the corresponding user has purchased the single-user mode project or the multi-user mode project), the information management device 300 sends an activation certificate to the communication device 200. The communication device 200 responds by operating in single-user mode upon identifying the activation certificate as corresponding to single-user mode. The communication device responds by operating in multi-user mode upon identifying the activation certificate as corresponding to multi-user mode.

In some embodiments, the communication device 200 executes an alcohol test program, to be compatible with the alcohol test device 100 and the information management device 300. The alcohol test program provides managers to log in to the information management device 300 through the management account for data management (including basic data management of the tested persons, management of alcohol test data, etc.). In some embodiments, after the alcohol test device 100 is paired with the communication device 200, the aforementioned operational control and information exchange can be executed. In some embodiments, managers can use the paired communication device 200 to perform data management. In some embodiments, managers can use other communication devices 201, 202 that are not paired with the alcohol test device 100 to log in to the information management device 300 for data management.

In some embodiments, the number of communication devices 200 that can be paired with the alcohol test device 100 is limited. The number of communication devices 200 that can be paired is restricted based on the activation certificate. For example, the activation certificate records a maximum pairing device value. Every time a new communication device 200 is paired with the alcohol test device 100, the alcohol test device 100 accumulates the number of paired communication devices 200. When a new communication device 200 wants to be paired with the alcohol test device 100, the communication device 200 and the alcohol test device 100 will confirm whether the number of paired devices has reached the maximum pairing device value. When the maximum pairing device value is reached, the pairing of the new communication device 200 with the alcohol test device 100 will be rejected. Conversely, the pairing will be allowed. The manager can operate the communication device 200 to unpair it from the alcohol test device 100.

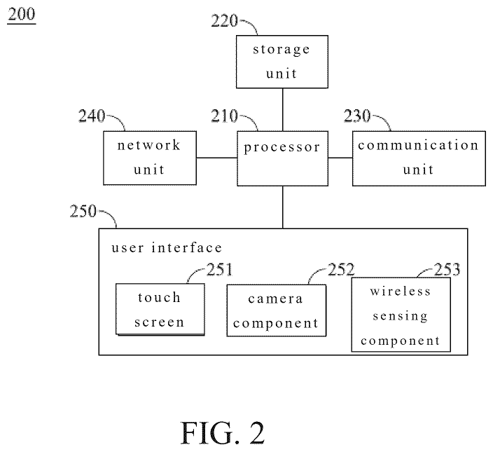

Refer to FIG. 2, which is a schematic diagram of the hardware architecture of an embodiment of the communication device 200 of the present invention. The communication device 200 includes a processor 210, a storage unit 220, a communication unit 230, a network unit 240, and a user interface 250. The processor 210 is coupled to the storage unit 220, the communication unit 230, the network unit 240, and the user interface 250 to control these components. The processor 210, for example, is a central processing unit (CPU), an application specific integrated circuit (ASIC), a microprocessor, etc. The storage unit 220 is a non-volatile storage medium (such as flash memory, hard disk, etc.) for storing the alcohol test program. The alcohol test program can be implemented as an application, an embedded program, or/and firmware. The communication unit 230 supports wired communication (such as USB) or/and wireless communication (such as Bluetooth) technologies to communicate with the alcohol test device 100. The network unit 240 supports wired network or wireless network communication technologies to connect to the information management device 300 through a network. The user interface 250 includes various input devices, including but not limited to a touch screen 251, a camera component 252, or/and a wireless sensing component 253, etc. In some embodiments, the communication device 200 is a mobile phone, a tablet computer, a laptop computer, or other electronic devices.

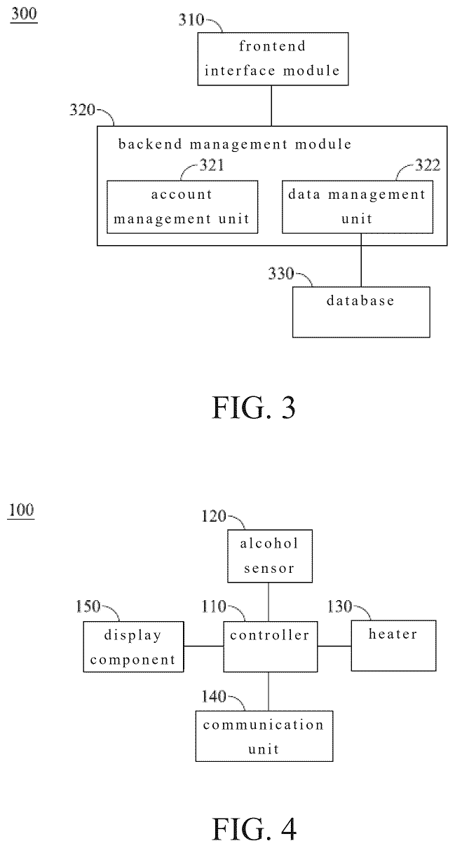

Refer to FIG. 3, which is a schematic diagram of the software architecture of an embodiment of the information management device 300 of the present invention. The information management device 300 includes a frontend interface module 310, a backend management module 320, and a database 330. The frontend interface module 310 is used to provide a front-end interface for the communication device 200, allowing users to browse alcohol test data and perform data management operations through the front-end interface. The backend management module 320 includes multiple management units, such as an account management unit 321 and a data management unit 322. The account management unit 321 is responsible for registering and logging in user accounts and other account management functions. The data management unit 322 is coupled to the database 330 to handle operations such as data addition, deletion, modification, and query in the database 330. In some embodiments, the information management device 300 is a cloud server or a local server, and its hardware composition is known to relevant technical personnel, so it will not be described here.

In some embodiments, the communication device 200 logs into the information management device 300 through a management account to pre-load (before executing the alcohol test cycle) the test subject data into the database 330 of the information management device 300. On the other hand, the identity data received by the alcohol test cycle from the alcohol test device 100 or the communication device 200 is the same or corresponded to the pre-loaded test subject data. Therefore, the information management device 300 can find the corresponding test subject data in the database 330 based on the identity data associated with the received alcohol test data, thereby determining the identity of the subject of the alcohol test cycle and adding the alcohol test data under the corresponding subject identity in the database 330.

Refer to FIG. 4, which is a schematic diagram of the hardware architecture of an embodiment of the alcohol test device 100 of the present invention. The alcohol test device 100 includes a controller 110, an alcohol sensor 120, a heater 130, a communication unit 140, and a display component 150. The controller 110 is coupled to the alcohol sensor 120, the communication unit 140, and the display component 150 to control these components. For example, the controller 110 is a central processing unit (CPU), an application specific integrated circuit (ASIC), a microprocessor, etc. The controller 110 also has built-in memory to store alcohol test data and information on the number of paired communication devices 200. In some embodiments, the controller 110 is coupled to external memory to store this information. The alcohol sensor 120 is used to conduct alcohol tests, and it can include one or more types of sensors, such as electrical, semiconductor, or infrared spectral types. Optionally, the alcohol test device 100 also includes a heater 130 to heat the alcohol sensor 120 to the appropriate temperature to stabilize the performance of the alcohol sensor 120 and improve measurement accuracy. The communication unit 140 supports wired communication or/and wireless communication technology to communicate with the communication unit 230 of the alcohol test device 100. The display component 150 is used to display specific patterns, which will be described in detail later.

In some embodiments, the heater 130 is preset to heat the alcohol sensor 120 at a fixed power. When the communication device 200 receives alcohol test data that does not meet the standards, it restarts another alcohol test cycle, causing the alcohol test device 100 to re-execute the alcohol test procedure. In this new alcohol test cycle, the alcohol test device 100, controlled by the communication device 200, changes an alcohol test operation.

The alcohol test operation can be to cause the heater 130 to use a variable power to heat the alcohol sensor 120, to adjust the sensitivity of gases that may interfere with the test results.

The alcohol test operation can also be changing the type of alcohol sensor 120 used, for example: originally using one type of alcohol sensor 120, and using two types of alcohol sensor 120 simultaneously in the new alcohol test cycle. Or, originally using the first type of alcohol sensor 120, and using the second type of alcohol sensor 120 in the new alcohol test cycle. In this way, by having different types of alcohol sensor 120 with varying sensitivities to interfering gases, the cause of the alcohol test data not meeting the standards can be identified as being due to the influence of interfering gases.

Alternatively, the alcohol test operation can be adjusting the alcohol test algorithm of the alcohol test device 100, such as adjusting the parameters in the algorithm.

In some embodiments, the heater 130 is preset to heat the alcohol sensor 120 with a heating time. However, after continuously executing the alcohol test cycle, the base temperature of the alcohol sensor 120 is increased, so it is no longer necessary to heat according to the preset heating time. Therefore, the communication device 200 reduces the heating time as the number of consecutive alcohol test cycles increases. By dynamically adjusting the heating time in this way, the execution time of the alcohol test cycle can be shortened.

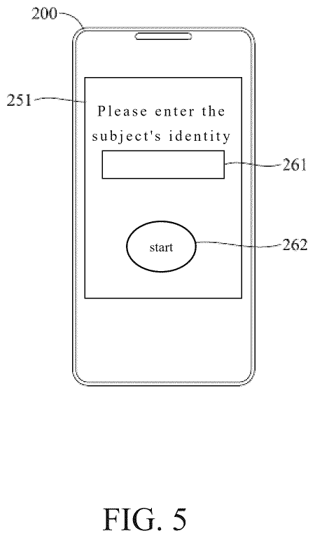

Refer to FIG. 5, which is a schematic diagram of an alcohol test operation according to an embodiment of the present invention. Corresponding to each alcohol test cycle (for example, before executing the alcohol test cycle), the user (who may be an administrator or a subject) operates the communication device 200 through the user interface 250 to input the identity data of the subject. In this case, the user interface 250 includes a touch screen 251, and the alcohol test program provides an input field 261 for the user to input the identity data through the touch screen 251. The identity data is data that can identify the subject's identity, such as a personal identifier, name, mobile phone number, email, etc. After inputting the identity data, in response to the user clicking the start button 262, the communication device 200 controls the alcohol test device 100 to start the alcohol test cycle. In some embodiments, the user can input the identity data of the subject for the current cycle after completing the alcohol test cycle.

In some embodiments, the user interface 250 includes a camera component 252, and the camera component 252 captures an image of the subject's face. The processor 210 executes a face recognition program to identify the subject's face image, thereby obtaining face recognition features (i.e., identity data) that can identify the subject's identity. In some embodiments, the face recognition program is executed during the subject's breathing process. That is to say, the processor 210 will execute the face recognition program based on whether the intensity of the breathing pressure (a pressure sensor may be configured in the breathing fluid channel to detect this) reaches a threshold, ensuring that the identity of the subject undergoing the alcohol test is verified.

In some embodiments, the user interface 250 includes a wireless sensing component 253. The wireless sensing component 253 supports Near-field communication (NFC) technology or Radio-frequency identification (RFID) technology, capable of reading an identification code (i.e., identity data) from a sensing tag.

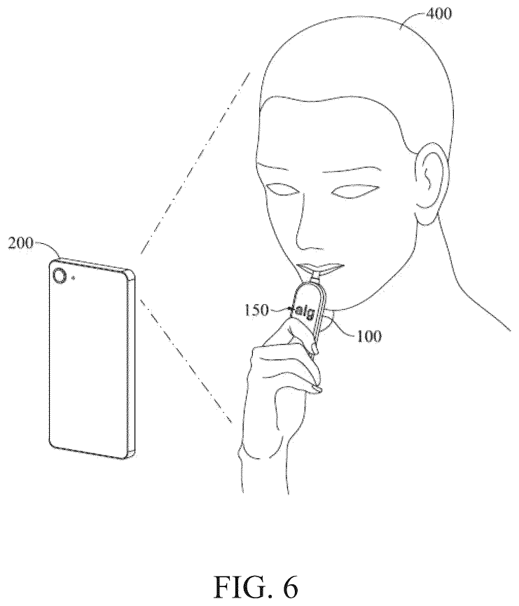

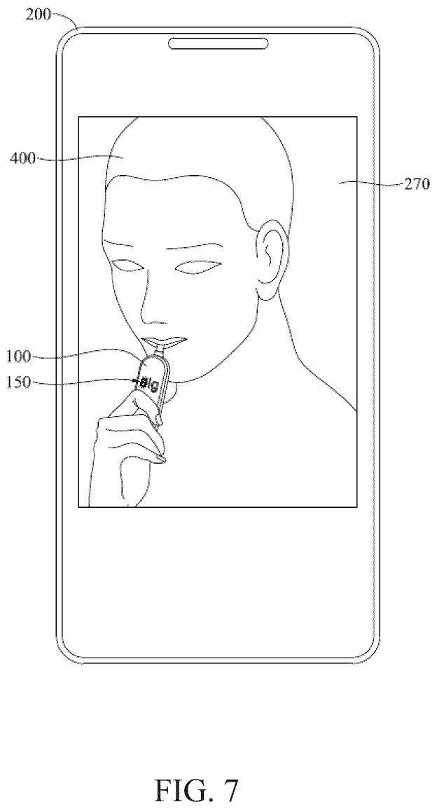

Refer to FIG. 6 and FIG. 7 together. FIG. 6 is a schematic diagram of another embodiment of the alcohol test operation of the present invention. FIG. 7 is a schematic diagram of an embodiment of the present invention capturing image 270 during the alcohol test operation. During the execution of the alcohol test cycle, the communication device 200 also captures an image 270 with the face of the subject 400 through the camera component 252. The processor 210 performs face recognition on the subject 400 in image 270 to generate a face recognition feature, and confirms whether the face recognition feature matches the test subject data associated with the identity data, and issuing an abnormal operation warning when they do not match. For example, if the user inputs the identity data of the first subject, but the face recognition feature of the second subject is identified during face recognition, since this face recognition feature does not match the test subject data associated with the identity data of the first subject, an abnormal situation of substituting blowing can be detected.

As shown in FIG. 6, in some embodiments, during the alcohol test cycle, the display component 150 of the alcohol test device 100 displays a selected image. This selected image is chosen from multiple images. The selected image is determined by the communication device 200. The camera component 252 of the communication device 200 takes images during the alcohol test cycle, and the captured images 270 show the subject 400 and the display component 150. The communication device determines whether the display content of the display component 150 in the image 270 matches the selected image. When they do not match, it indicates that the alcohol test device 100 in the image 270 is not bound to the communication device 200. The alcohol test device 100 that is actually bound to the communication device 200 is located outside the image 270 and is blown by someone else, thereby issuing an abnormal operation warning.

As shown in FIG. 6, in some embodiments, the display component 150 includes a plurality of lighting units, wherein these lighting units have distinguishable visual characteristics, such as being distinguished by lighting color, lighting intensity, or/and lighting shape. For example, as shown in FIG. 6, the lighting units have three different letter shapes, a, 1, g, and can be distinguished by lighting shape. The aforementioned multiple patterns are selected from these lighting units, a portion of the lighting units, or all the lighting units. When a specific lighting combination is selected, the aforementioned selected image is generated. As shown in FIG. 7, this is a schematic diagram of the selected image in one embodiment of the present creation. Here, the lighting units with the shape of the a letter are lit, and the other lighting units are not lit.

In some embodiments, the display component 150 is implemented on a screen and can display content such as a barcode (e.g., a one-dimensional barcode or a two-dimensional barcode) or a string that is identifiable by an image, such as the selected image.

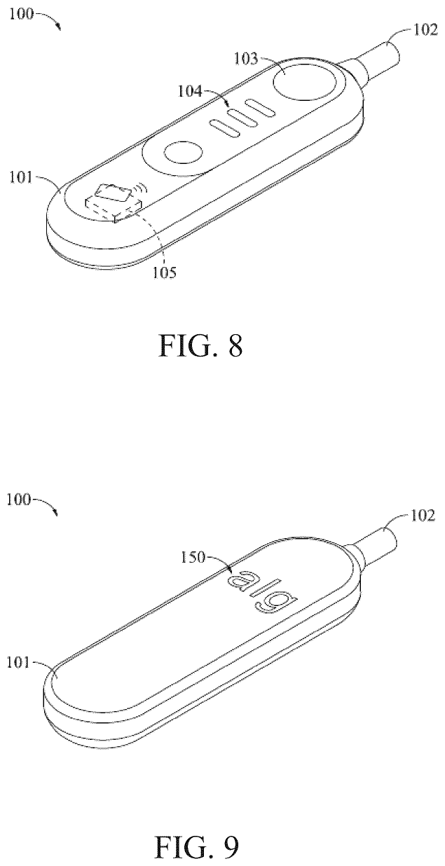

Refer to FIG. 8 and FIG. 9 together, which are perspective views (1) (2) of an embodiment of the alcohol test device 100 of the present invention. The alcohol test device 100 includes a main body 101 and a blower mouthpiece 102. The interior of the main body 101 is provided with a controller 110, an alcohol sensor 120, a heater 130, and a communication unit 140 (not shown in the figure). The interior of the main body 101 is provided with a fluid channel for the gas introduced by the blower mouthpiece 102 to flow towards the alcohol sensor 120 (not shown in the figure). The first surface of the main body 101 is provided with a switch 103, which is used for being pressed by the user to start the alcohol test device 100. The first surface of the main body 101 is also provided with an alcohol test information unit 104, which displays alcohol test information. Here, the degree of alcohol test information is presented using different color indicators (such as red, yellow, and green). When the measured alcohol test value is within the first range, the controller 110 controls the alcohol test information unit 104 to display the first color indicator; when the measured alcohol test value is within the second range, the controller 110 controls the alcohol test information unit 104 to display the second color indicator; and so on. In some embodiments, the alcohol test information can be an alcohol test value. The second surface of the main body 101, opposite to the first surface, is provided with a display component 150, which allows the camera component 252 of the communication device 200 to take pictures when the subject blows air.

In some embodiments, the alcohol test device 100 further includes a wireless sensing component 105. The wireless sensing component 105 supports near-field communication (NFC) technology or radio-frequency identification (RFID) technology, capable of reading an identification code (i.e., identity data 108) in a sensing tag.

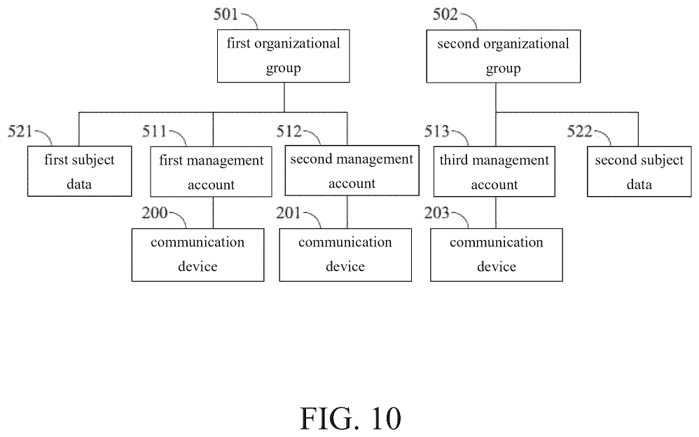

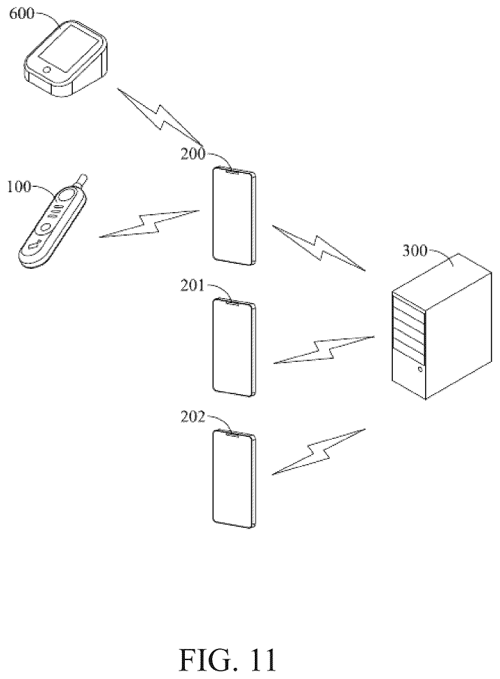

Refer to FIG. 1 and FIG. 10 together. FIG. 10 is a schematic organizational management diagram of an embodiment of the present invention. The communication devices 200 to 203 can log into the information management device 300 through different management accounts. For example, logging into the information management device 300 with the first management account 511 on the communication device 200; logging into the information management device 300 with the second management account 512 on the communication device 201; logging into the information management device 300 with the third management account 513 on the communication device 203. Some management accounts can be assigned to a organizational group, while others can be assigned to a different organizational group. For example, the first management account 511 and the second management account 512 are assigned to the first organizational group 501, while the third management account 513 is assigned to the second organizational group 502. The previously preloaded test subject data into the information management device 300 can be executed through multiple management accounts. The test subject data loaded into the information management device 300 through management accounts will be assigned to the organizational group associated with that management account. For example, the first subject data 521 loaded by the first management account 511 or the second management account 512 will be assigned to the first organizational group 501; the third test subject data 523 loaded by the third management account 513 will be assigned to the second organizational group 502. In this way, management accounts assigned to the same organizational group can load the test subject data under the same organizational group, without being limited to a single management account for loading, and can be operated simultaneously by multiple management accounts, to save time.

In some embodiments, an organizational group can be a company, a subsidiary, a factory, or a transport station, or another organizational group under those institution.

In some embodiments, referring to FIG. 1 and FIG. 10 together, when the information management device 300 detects that the alcohol test data corresponding to the first subject data 521 belonging to the first organizational group 501 does not meet the standards, it sends a notification message to the communication device 200˜201 corresponded to the management account (such as the first management account 511 or/and the second management account 512) in the first organizational group 501. Thus, the manager can be informed of the abnormal alcohol test status of the subjects within the same organizational group. The notification message can be sent through push, SMS, phone, or/and email, etc. Similarly, when the alcohol test data of the second subject data 522 belonging to the second organizational group 502 does not meet the standards, the information management device 300 will also send a notification message to the communication device 203 corresponded to the management account (such as the third management account 513) in the second organizational group 502.

In some embodiments, some subjects will not always perform the alcohol test within the same organizational group, for example, a truck driver transporting from A shipping station to B shipping station, where alcohol tests are conducted at both A and B shipping stations. When the alcohol test data corresponded to the first subject 521 does not meet the standards, and this non-conforming alcohol test data is generated by the alcohol test cycle initiated by the communication device 203 corresponded to the management account belonging to the second organizational group 502 (such as the third management account 513), the information management device 300 also sends a notification message to the communication device 203 corresponded to the management account belonging to the second organizational group 502 (such as the third management account 513). For instance, when the truck driver regulated at A shipping station detects non-conforming alcohol test data at B shipping station, the information management device 300 will notify the manager at B shipping station to further verify whether the truck driver indeed has non-conforming alcohol test values and take appropriate measures for control.

Refer to FIG. 11, which is a schematic diagram of the structure of another embodiment of the alcohol test management system of the present invention. The alcohol test management system can further include a blood pressure measurement device 600, which is electrically connected to the communication device 200. The blood pressure measurement device 600 is used to conduct blood pressure tests and to generate a blood pressure data respectively after completing each blood pressure test cycle. Similar to the alcohol test cycle, each blood pressure test cycle is controlled by the communication device 200 and corresponds to a corresponding alcohol test cycle. After the communication device 200 receives the blood pressure data corresponded to the blood pressure test cycle, it associates the blood pressure data with the identity data of the corresponding alcohol test cycle. The information management device 300, after receiving the blood pressure data associated with the identity data from the communication device 200, records the blood pressure data in the database 330 and conducts statistical analysis. Through statistical analysis (such as taking the average or median, etc.), the blood pressure performance of the corresponding subject can be determined.

The manner in which the identity data is associated with the alcohol test data/blood pressure data, as described above, can be, for example, by adding the identity data to the alcohol test data/blood pressure data or by associating information related to the identity data.

According to the alcohol test management system of the present invention, the operation mode of the communication device 200 can be determined by the activation certificate sent from the information management device 300, whether it operates in single-user mode or multi-user mode. Users can change the operation mode of the alcohol test device 100 to single-user or multi-user mode after purchasing the alcohol test device 100. Therefore, the alcohol test device 100 does not need to distinguish between single-user and multi-user scenarios by separate hardware, which is beneficial for product production, manufacturing, and sales. Additionally, since the alcohol test cycle is started by the communication device 200, the subject's identity can be verified through the communication device 200 during the alcohol test process, providing anti-fraud effects.

What is claimed is:

1. An alcohol test management system, comprising:
an alcohol test device, respectively generating an alcohol test data after completing each alcohol test cycle;
a communication device, electrically connected to the alcohol test device, to control the alcohol test device in starting each of the alcohol test cycle, and to receive the alcohol test data generated during each of the alcohol test cycle from the alcohol test device, and associating each of the alcohol test data with an identity data, the communication device being configured in a single-user mode or a multi-user mode, wherein in the single-user mode, the identity data associated with each alcohol test data received is bound to the communication device, and in the multi-user mode, the identity data associated with each alcohol test data received is received by the alcohol test device or the communication device corresponding to the alcohol test cycle; and
an information management device, electrically connected to the communication device, to record each of the alcohol test data received from the communication device associated with the identity data, and to send an activation certificate to the communication device, wherein the communication device operates in the single-user mode upon identifying that the activation certificate corresponds to the single-user mode, and the communication device operates in the multi-user mode upon identifying that the activation certificate corresponds to the multi-user mode.

2. The alcohol test management system as claimed in claim 1, wherein each of the identity data is the same or corresponds to a test subject data, the communication device logs into the information management device through a management account, and pre-loads the test subject data into the information management device.

3. The alcohol test management system as claimed in claim 2, wherein the communication device are multiple, each of the communication devices logs into the information management device through a plurality of management accounts which are different, the plurality of management accounts are in an organizational group, and the test subject data loaded through the plurality of management accounts into the information management device is attributed to the organizational group.

4. The alcohol test management system as claimed in claim 2, wherein the communication device is two, the management account of one of the communication devices is in a first organizational group, and the management account of the other of the communication devices is in a second organizational group, wherein when the information management device detects that the alcohol test data corresponding to the test subject data belonging to the first organizational group does not meet standard, sending a notification message to the communication device corresponded to the management account in the first organizational group.

5. The alcohol test management system as claimed in claim 4, wherein the alcohol test data that does not meet standard is generated by the alcohol test cycle started by the communication device corresponded to the management account in the second organizational group, and the information management device also sends another notification message to the communication device corresponded to the management account in the second organizational group.

6. The alcohol test management system as claimed in claim 1, wherein the alcohol test device comprises an alcohol sensor and a heater for heating the alcohol sensor, the heater is preset to heat at a fixed power, when the communication device receives the alcohol test data that does not meet standard, restarts another alcohol test cycle, and controls the alcohol test device to change an alcohol test operation, the alcohol test operation comprises: the heater heating at a variable power, changing a type of alcohol sensor used, or adjusting the alcohol test algorithm of the alcohol test device.

7. The alcohol test management system as claimed in claim 1, wherein the alcohol test device comprises an alcohol sensor and a heater for heating the alcohol sensor, the heater heats the alcohol sensor with a heating time, the communication device controls the alcohol test device to reduce the heating time as a number of consecutive executions of the alcohol test cycle increases.

8. The alcohol test management system as claimed in claim 1, wherein the alcohol test device comprises a display component, for displaying a selected image from a plurality of images during the alcohol test cycle, the selected image is determined by the communication device, wherein the communication device comprises a camera component, for displaying a subject and the display component in an image captured during the alcohol test cycle, the communication device determines whether a displayed content of the display component in the image matches the selected image, and the communication device issues an abnormal operation warning when not matching.

9. The alcohol test management system as claimed in claim 8, wherein the communication device further performs face recognition on the subject in the image to generate a face recognition feature, and confirms whether the face recognition feature corresponds to the test subject data associated with the identity data, issuing another abnormal operation warning when not consistent.

10. The alcohol test management system as claimed in claim 1, further comprising:
a blood pressure measurement device, electrically connected to the communication device, to generate a blood pressure data respectively after completing each blood pressure test cycle, wherein the communication device controls the blood pressure measurement device to start each of the blood pressure test cycle corresponded to each of the alcohol test cycle, and receives the blood pressure data from the blood pressure measurement device to associate each of the blood pressure data with the identity data which is corresponding, the information management device records and statistically analyzes each of the blood pressure data received from the communication device.
